(19) United States
(12) Patent Application Publication
(10) Pub. No.: US 2022/0226155 A1
(43) Pub. Date: Jul. 21, 2022

(54) VISOR ASSEMBLY AND METHOD FOR SHIELDING A WEARER'S FACE

(71) Applicant: InkSmith Ltd., Waterloo (CA)

(72) Inventors: Douglas James Braden, Waterloo (CA); Jeremy Richard Hedges, Waterloo (CA)

(21) Appl. No.: 17/579,111

(22) Filed: Jan. 19, 2022

Related U.S. Application Data
(60) Provisional application No. 63/139,148, filed on Jan. 19, 2021.

(51) Int. Cl. A61F 9/02; A41D 13/11; A62B 18/08

(52) U.S. Cl. CPC A61F 9/029; A61F 9/026; A62B 18/084; A41D 13/1184

(57) ABSTRACT

Provided are a visor assembly, visor, and a method for protecting a wearer's face. The visor assembly comprises a visor configured to connect to a disposable face mask, the visor comprising: a first surface that is substantially flat when the visor is not connected to the disposable face mask, wherein the first surface is substantially curved when the visor is connected to the disposable face mask and worn; a second surface that is substantially flat when the visor is not connected to the disposable face mask, wherein the second surface is substantially curved when the visor is connected to the disposable face mask and worn; two protruding members that protrude substantially in parallel to the first surface towards the disposable face mask, each member comprising at least one slit through which at least one fastening element of the disposable face mask may pass at least once.

### TECHNICAL FIELD

The embodiments disclosed herein relate to visor assemblies, visors, and methods of face protection, and more particularly to visor assemblies for use in personal protective equipment (hereinafter “PPE”).

### INTRODUCTION

Medical personnel have long required PPE to protect themselves from contagious situations, in the case of illness or emergency situations involving exposure to bodily fluid. Given the contagious nature of many medical conditions, medical personnel cannot effectively treat patients if they run the risk of transmitting infectious diseases from one patient to the next. Furthermore, when medical personnel themselves take ill, fewer resources are available to treat their patients. It is of paramount importance that efficient and efficacious means of preparing and providing PPE be established.

In the present pandemic situation, civilian populations also require access to PPE, motivating individuals, private corporations, health organizations, and government to find cost-effective solutions. Businesses cannot thrive when customers risk transmitting disease among themselves and among staff, while individuals will not be and will not feel safe attending schools, travelling to workplaces, and generally spending time outside their own homes without appropriate PPE.

While conventional face masks provide for protection of the nose and mouth and associated breathing and internal airway passages, personnel in potentially contagious or otherwise unclean environments or circumstances may desire or require greater protection for vulnerable areas of the face. Accordingly, the present disclosure provides for visors configured to easily attach and remain securely fastened to a face mask on a wearer's face. The resultant combination of visor and face mask (e.g., conventional surgical mask) is herein termed a “visor assembly”. Such visor assemblies preferably cover the entirety of the front of the wearer's face, especially the eyes, nose, and mouth.

### SUMMARY

The present application discloses embodiments that relate to visors and visor assemblies as personal protective equipment and methods of manufacturing and using same.

In accordance with one embodiment, a visor assembly to be worn as personal protective equipment about a wearer's face is provided. The visor assembly includes a visor configured to connect to a disposable face mask, the visor including a first surface that is substantially flat when the visor is not connected to the disposable face mask, and the first surface is substantially curved when the visor is connected to the disposable face mask and worn; a second surface that is substantially flat when the visor is not connected to the disposable face mask, and the second surface is substantially curved when the visor is connected to the disposable face mask and worn; two protruding members that protrude substantially in parallel to the first surface towards the disposable face mask, each member including at least one slit through which at least one fastening element of the disposable face mask may pass at least once; and a face mask including the at least one fastening element.

When the fastening elements are passed through the slits of the members of the visor, the visor may be attached to the disposable face mask when worn after the fastening elements of the disposable face mask have passed through the slits, the wearer may adjust the face mask component of the visor assembly about the wearer's own face, and the wearer may adjust the visor component of the visor assembly about the wearer's own face by moving the visor component about the fastening elements.

In an aspect, when in use, the visor is sufficiently flexible to mold to the wearer's face, and when in use the visor deforms no more than necessary for the at least one fastening element to remain substantially straight. When not in use the visor assumes its original shape, and the visor is made of transparent material.

In another aspect, only the first surface of the visor is made of transparent material.

In another aspect, the second surface includes a cutout region to accommodate a wearer's nose.

In another aspect, the cutout region is sufficiently flexible to fold around the wearer's nose.

In another aspect, the visor further includes third surfaces adjacent to the first surface that wrap around sides of the wearer's face when the visor is in use.

In another aspect, the visor is made of a recyclable material.

In another aspect, the visor may be separated from the visor assembly and cleaned for reuse.

In another aspect, the third surfaces include a compound angle, the first surface includes a dome portion as an upper edge, the members are canted outwards, and the members rest atop the mask.

In another aspect, the slits further include at least one cut to hold the at least one fastening element in place.

In another aspect, there is provided a kit including the visor and face mask herein described.

In accordance with another embodiment, there is provided a visor configured to connect to a disposable face mask to be about a wearer's face. The visor includes a first surface that is substantially flat when the visor is not connected to the disposable face mask and is substantially curved when the visor is connected to the disposable face mask and worn; a second surface that is substantially flat when the visor is not connected to the disposable face mask and is substantially curved when the visor is connected to the disposable face mask and worn; two protruding members that protrude substantially in parallel to the first surface towards the disposable face mask, each member including at least one slit through which at least one fastening element of the disposable face mask may pass at least once.

The visor cannot be detached from the disposable face mask when worn after the fastening elements of the disposable face mask have passed through the slits, and the wearer may adjust the visor about the wearer's own face without affecting the face mask by pivoting and/or sliding the visor about the fastening elements.

In an aspect, when in use the visor is sufficiently flexible to mold to the wearer's face, and when in use the visor deforms no more than necessary for the at least one fastening element to remain substantially straight. When not in use, the visor assumes its original shape, and the visor is made of transparent material.

In another aspect, only the first surface of the visor is made of transparent material.

In another aspect, the second surface includes a cutout region to accommodate a wearer's nose.

In another aspect, the cutout region is sufficiently flexible to fold around the wearer's nose.

In another aspect, the visor further includes third surfaces adjacent to the first surface that wrap around sides of the wearer's face when the visor is in use.

In another aspect, the visor is made of a recyclable material.

In another aspect, the visor may be separated from the disposable face mask and cleaned for reuse.

In another aspect, the third surfaces include a compound angle, the first surface includes a dome portion as an upper edge, the members are canted outwards, and the members rest atop the mask.

In another aspect, the slits further include at least one cut to hold the at least one fastening element in place.

In accordance with another embodiment, there is provided a method of shielding a wearer's face from biological hazards. The method includes passing at least one fastening element of a face mask through slits of a visor to assemble a visor assembly, with two protruding members of the visor protruding substantially in parallel to a first surface of the visor towards the disposable face mask, and each member includes at least one slit through which the at least one fastening element of the disposable face mask may pass at least once. The method further includes pressing the visor assembly against their own face, and attaching the at least one fastening element of the face mask now passed through the slits of the visor around the wearer's own head,

The visor may not be detached from the face mask when worn after the at least one fastening element has passed through the slits. The wearer may adjust the face mask component of the visor assembly about the wearer's own face, and the wearer may adjust the visor component of the visor assembly about the wearer's own face without affecting the face mask component by pivoting and/or sliding the visor component about the at least one fastening element.

In an aspect, when in use the visor is sufficiently flexible to mold to the wearer's face, and when in use the visor deforms no more than necessary for the at least one fastening element to remain substantially straight. When not in use the visor assumes its original shape, and the visor is made of transparent material.

In another aspect, only the first surface of the visor is made of transparent material.

In another aspect, the second surface includes a cutout region to accommodate a wearer's nose.

In another aspect, the cutout region is sufficiently flexible to fold around the wearer's nose.

In another aspect, the visor further includes third surfaces adjacent to the first surface that wrap around sides of the wearer's face when the visor is in use.

In another aspect, the wearer attaches the at least one fastening element of the face mask around the wearer's own head before pressing the visor assembly against the wearer's own face.

In another aspect, the visor is made of a recyclable material.

In another aspect, separating the visor from the visor assembly and cleaning the visor for reuse.

In another aspect, the third surfaces include a compound angle, the first surface includes a dome portion as an upper edge, the members are canted outwards, and the members rest atop the mask.

In another aspect, the slits further include at least one cut to hold the at least one fastening element in place.

In accordance with another embodiment, there is provided a method of manufacturing a visor configured to connect to a disposable face mask to be worn about a wearer's face. The method includes preparing optically clear plastic according to known chemical processes, using a sheet extrusion line to produce sheets of the optically clear plastic, and using any of laser cutting, rotary die cutting, die cutting, stamping press, or other technology to cut the sheets into the shape of visors. The visors include a first surface that is substantially flat when the visor is not connected to the disposable face mask, and the first surface is substantially curved when the visor is connected to the disposable face mask and worn; a second surface that is substantially flat when the visor is not connected to the disposable face mask, and the second surface is substantially curved when the visor is connected to the disposable face mask and worn. The visor further includes two protruding members that protrude substantially in parallel to the first surface towards the disposable face mask, each member including at least one slit through which at least one fastening element of the disposable face mask may pass at least once; the visor cannot be detached from the disposable face mask when worn after the at least one fastening element of the disposable face mask have passed through the slits, and the wearer may adjust the visor about the wearer's own face without affecting the face mask by pivoting and/or sliding the visor about the at least one fastening element.

In an aspect, stamping the plastic includes stamping multiple sheets of the plastic at once.

In another aspect, when in use the visor is sufficiently flexible to mold to the wearer's face, and when in use the visor deforms no more than necessary for the at least one fastening element to remain substantially straight. When not in use the visor assumes its original shape, and the visor is made of transparent material.

In another aspect, only the first surface of the visor is made of transparent material.

In another aspect, the second surface includes a cutout region to accommodate a wearer's nose.

In another aspect, the cutout region is sufficiently flexible to fold around the wearer's nose.

In another aspect, the visor further includes third surfaces adjacent to the first surface that wrap around sides of the wearer's face when the visor is in use.

In another aspect, the visor is made of a recyclable material.

In another aspect, the visor may be separated from the disposable face mask and cleaned for reuse.

In another aspect, the third surfaces include a compound angle, the first surface includes a dome portion as an upper edge, the members are canted outwards, and the members rest atop the mask.

In another aspect, the slits further include at least one cut to hold the at least one fastening element in place.

Other aspects and features will become apparent, to those ordinarily skilled in the art, upon review of the following description of some exemplary embodiments.

### BRIEF DESCRIPTION OF THE DRAWINGS

The drawings included herewith are for illustrating various examples of articles, methods, and apparatuses of the present specification. In the drawings:

FIG. 1 is an angled side view of an embodiment of the visor of the present disclosure in use, affixed to a disposable face mask to form an embodiment of the visor assembly of the present invention;

FIG. 2 is a visor for shielding a wearer's face, according to an embodiment;

FIG. 3 is a perspective view of the visor of FIG. 2;

FIG. 4 is a visor for shielding a wearer's face, in accordance with another embodiment;

FIG. 5 is a visor for shielding a wearer's face, in accordance with another embodiment;

FIGS. 6 to 10 are perspective, side perspective, top, bottom perspective and side views, respectively of a visor for shielding a wearer's face, in accordance with another embodiment;

FIG. 11 is a flow diagram of a method of shielding a wearer's face from biological hazards;

FIG. 12 is a flow diagram of a method of manufacturing a visor configured to connect to a disposable face mask;

FIG. 13 is an enlarged view of part of the visor and visor assembly of FIG. 1; and

FIG. 14 is another view of the visor assembly of FIGS. 1 and 13.

### DETAILED DESCRIPTION

Various apparatuses or processes will be described below to provide an example of each claimed embodiment. No embodiment described below limits any claimed embodiment and any claimed embodiment may cover processes or apparatuses that differ from those described below. The claimed embodiments are not limited to apparatuses or processes having all of the features of any one apparatus or process described below or to features common to multiple or all of the apparatuses described below.

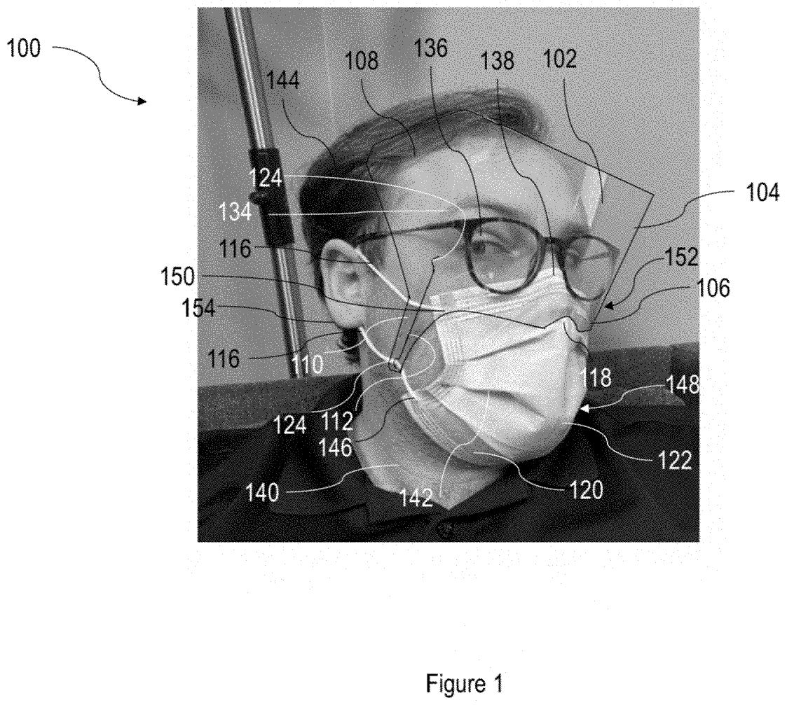

Referring now to FIG. 1, shown therein is an embodiment of a visor assembly 100 for protecting a wearer's face 134, in accordance with an embodiment. The visor assembly 100 includes a visor 102 for protecting eyes 136 of the wearer 140 and a face mask 120 for covering a mouth 142 and a nose 138 of the wearer 140.

A black outline approximately corresponding to the shape of the visor 102 is provided in FIG. 1 for ease of viewing. A similar outline is provided corresponding to the shape of one of the slits 112.

The mask 120 includes main body 122 for covering the mouth 142 and the nose 138 of the wearer 140. The main body 122 of the mask 120 may be a disposable surgical mask. The main body 122 may also be a respirator The main body 122 may also be a cloth mask. The main body 122 may be any kind of face covering suitable for covering the mouth 142 and the nose 138 of the wearer 140.

The mask 120 further includes one or more fastening elements 116 for securing the mask 120 and eventually the visor assembly 120 to the face 134 of the wearer 140.

The visor assembly 100 includes the visor 102 for protecting the eyes 136 of the wearer 140. The visor 102 includes a first surface 104 for curving about the face 134 of the wearer 140, a second surface 106 for curving about the face 134 of the wearer 140, and third surfaces 108 for wrapping around a wearer's head 144 or face 134.

Advantageously, the visor 102 may be used in combination with any face mask 120. Whereas the mask 120 covers the nose 138 and mouth 142 of the wearer 140, the visor 102 covers the eyes 136 of the wearer 140. Accordingly, where the mask 120 has fastening elements 116 for securing the mask about the head 144 of the wearer, the combined visor assembly 100 may protect the entirety of the face 134 of the wearer 140.

The first and second surfaces 104, 106 are substantially flat when the visor 102 is not connected to the face mask 120. Advantageously, multiple visors 102 may easily be stacked together for storage and/or shipping. The first and second surfaces 104, 106 are substantially curved when the visor 102 is connected to the face mask 120 and the visor assembly 100 is worn. Accordingly, the first and second surfaces 104, 106 may be molded to the face 134 of the wearer 140.

The first and second surfaces 104, 106 may be sufficiently flexible both to remain flat when not worn by the wearer 140 and to mold to the wearer's face 134 when in use. The first and second surfaces 104, 106 may then be sufficiently flexible to assume its original shape when not in use. Such flexibility advantageously ensures that a visor 102 is not permanently curved or deformed upon use by a wearer 140 and may be easily stored after use as previously described herein.

The third surfaces 108 wrap around sides of the wearer's own face 134 or head 144 when the visor 102 is in use, as shown in FIG. 1. Such wrapping advantageously ensures greater protection for the wearer's own face 134 and/or head 144 when the visor 102 is in use. Such wrapping may further help to secure the visor 102 and accordingly the visor assembly 100 when in use by the wearer 140.

In FIG. 1, fastening elements 116 connect to the mask 120 at each of bottom-left, bottom-right, top-left, and top-right corners 146, 148, 150, and 152, respectively, of the face mask 120 (directions are relative to FIG. 1 itself). That the at least one fastening element 116 connect to the face mask 120 across multiple corners 146, 148, 150, and 152 may advantageously help distribute tension among the at least one fastening element 116. The proper distribution of tension among the at least one fastening element 116 may make the at least one fastening element 116 less likely to stretch or break, which in turn may make the face mask 120 and accordingly the visor assembly 100 less likely to fall off the face 134 of the wearer 140. That the at least one fastening element 116 connect to the face mask 120 across multiple corners 146, 148, 150, and 152 may further advantageously cause the mask 120 and accordingly the visor assembly 100 to remain in place on the wearer's face 134 and/or head 144 despite the stretching or breaking of one or more fastening elements 116.

The at least one fastening element 116 may include earloops 116. Such earloops may be easy for a wearer 140 to secure about ears 154 of the wearer 140. Such earloops 116 may further act to properly distribute tension among two or more corners 146, 148, 150, and 152 as previously described. Such earloops 116 may be made of fabric, elastic, or other materials or a combination thereof. Elastic earloops 116 may be able to stretch and so properly distribute tension as previously described while returning to their original shape.

The earloops 116 may connect to top-left corner 150 and bottom-left corner 146 and to top-right corner 152 and bottom-right corner 148. The earloops 116 may instead connect top-left corner 150 to top-right corner 152 and bottom-left corner 146 to bottom-right corner 148. Such earloops 116 may instead be termed headbands 116. Other combinations may be possible.

The at least one fastening element 116 may include individual cords or strings 116.

The at least one fastening element 116 connected to any one of the corners 146, 148, 150, and 152 may connect to one or more fastening elements 116 connected to any one of the corners 146, 148, 150, and 152 (for example, by tying the individual cords or strings 116 together into a knot). Such a combination of multiple possible connections may advantageously allow a wearer 140 to adjust and secure the mask 120 in a variety of ways in order to ensure protection of the wearer's own face 134.

The visor 102 further includes two protruding members 110. The two members 110 protrude substantially in parallel to the first surface 104 towards the mask 120. Each member 110 includes at least one slit 112. Advantageously, the members 110 protrude towards the mask 120 in order to reach the bottom corners 146 and 148 of the mask 120. Where the members 110 extend fully towards and past the bottom corners 146 and 148, the at least one fastening element 116 may be passed through the slits 112 without unduly stretching or deforming the at least one fastening element 116. Such passage through the slits 112 may cause the mask 120 and accordingly the visor assembly 100 to more securely and easily remain in place on the face 134 and/or about the head 144 of the wearer 140, thereby better protecting the wearer 140. The elastic nature of 116 allows for a range of positions of placement of the visor 102 on the wearer's face 134 in order to accommodate a variety of faces 134 and preferences for wear.

The members 110 and bottom of the second surface 106 may rest on top of the mask 120. The members 110 and bottom of the second surface 106 when so resting may help retain the mask 120 in place about the wearer's own face 134. This configuration may improve the functionality of the mask 120 in creating and retaining a better seal between the mask 120 and the wearer's own face 134.

One of the at least one fastening element 116 may pass through a slit 112 one or more times. For example, where a fastening element 116 is an individual cord or string as previously described, each fastening element 116 may pass through its corresponding slit 112 once before being connected, for example through tying a knot, to another fastening element 116 that has passed through its corresponding slit 112. Alternately, where the fastening element 116 is an earloop 116 (such as in FIG. 1), the fastening element 116 may pass through its corresponding slit 112 twice (such as through cuts 124).

The visor 102 further includes a region 118 to accommodate the nose of a wearer of the visor assembly 100. The material of the visor 102 may be flexible enough such that the region 118 is sufficiently flexible to accommodate a wearer's nose without discomfort or undue pressure.

The slits 112 may be between 0.1 and 5 cm in length. The slits 112 may be between 0.1 and 3 cm in length. The slits 112 may be between 0.1 and 1 cm in length. The slits 112 may be between 0.5 and 1 cm in length. The slits 112 may be between 0.1 and 0.5 cm in length.

The members 110 may be between 1 and 10 cm in length. The members 110 may be between 3 and 7 cm in length. The members 110 may be between 4 and 6 cm in length.

The slits 112 may be positioned between 1 and 10 cm away from a distal edge of the members 110. The slits 112 may be positioned between 3 and 7 cm away from the distal edge of the members 110. The slits 112 may be positioned between 4 and 6 cm away from the distal edge of the members 110.

The slits 112 may be substantially straight and parallel to the first surface 104. The slits 112 may be substantially straight and perpendicular to the first surface 104. The slits 112 may be substantially straight and may be at an angle (not shown) that is neither parallel nor perpendicular relative to the first surface 104.

The slits 112 may be substantially curved and convex relative to an interior side edge of each member 110. The slits 112 may be substantially curved and concave relative to an interior side edge of each member 110.

Advantageously, the visor 102 may be capable of cleaning and reuse after separation from the mask 120 to effect disassembly of the visor assembly 100. By contrast, conventional means of attaching the visor 102 to the mask 120, such as welding, rather than the use of fastening elements 116 and slits 112 as herein described, are not susceptible to such cleaning and reuse as the visor 102 may accordingly not be easily separable from the mask 120.

Advantageously, the visor 102 may be made of a recyclable material.

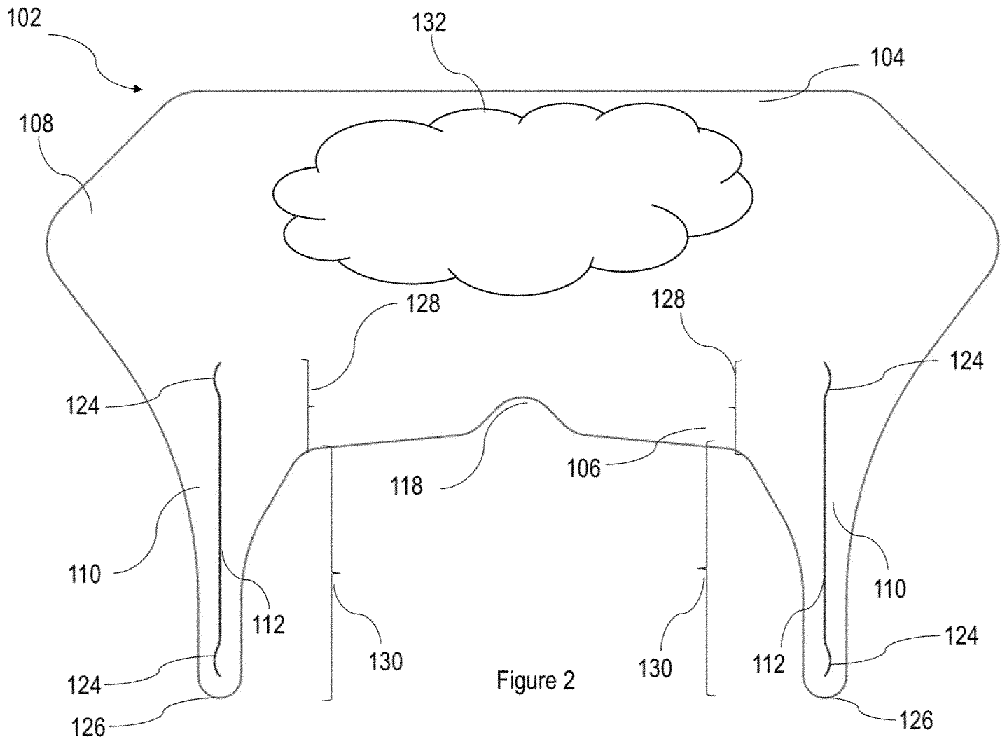

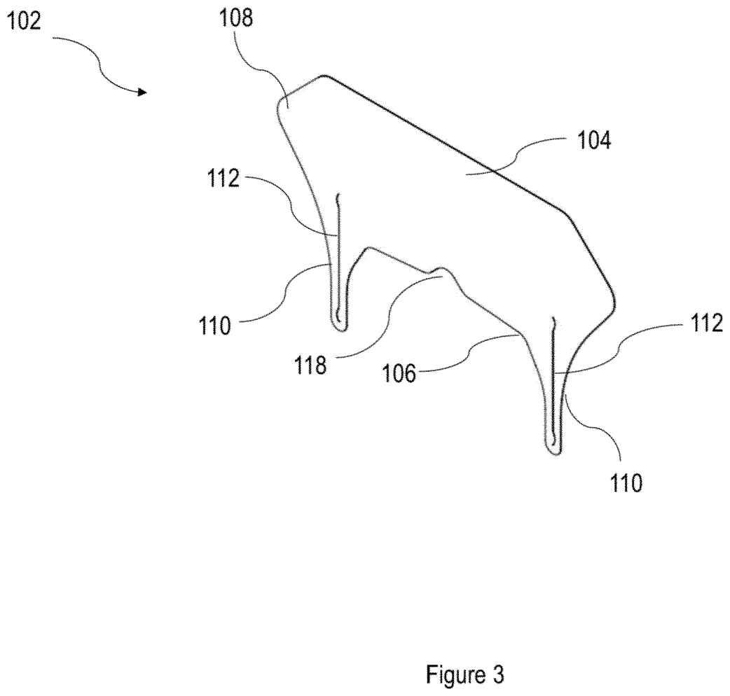

Referring now to FIGS. 2 and 3, shown therein is a block diagram of the visor 102 of FIG. 1 not connected to the face mask 120. In FIG. 2, like references denote like features.

Certain features of the visor 120 may be clearer in FIG. 2 than in FIG. 1. For example, the protruding members 110 herein protrude substantially in parallel to the first surface 104 of the visor 102. The members 110 include slits 112 through which a fastening element 116 may pass one or more times.

The members 110 further include rounded edges 126. With such rounded edges 126, the manufacturability of the visor 102 may be increased greatly. Furthermore, sharp edges may catch on other fabric and/or PPE and may inadvertently cause breaks in the skin of the wearer 140, which are vectors for infection. Such rounded edges 126 may advantageously avoid causing the wearer physical discomfort or damaging the face mask 120, as might otherwise be the case with sharp or otherwise straight, flat edges on the members 110.

In FIG. 2, the slits 112 begin within the members 110 but continue upwards past the members 110 and into the second surface 106. The slits 112 may further extend upwards past the second surface 106 and into the first surface 104.

The slits 112 further include the cuts 124, which may be at the top and/or bottom of the slits 112 as shown or elsewhere along the slits 112, for holding the at least one fastening element 116. These cuts may be in the shape of curves, as shown in FIG. 1, or in a D′ or rectangular shape. The cuts 124 may retain the at least one fastening element 116 at the top and bottom of the slits 112 when the at least one fastening element 116 are passed through the slits 112 by the wearer. For example, where the at least one fastening element 116 are earloops 116 of the disposable face mask 120, the cuts 124 may ensure that top and bottom portions of the earloops 116 do not tangle or otherwise migrate to the middle of the slit 112, which might cause discomfort for the wearer 140 and might result in the face mask 120, the visor 102, and/or the entire visor assembly 100 not remaining on the wearer's face 134 and/or about the wearer's head 144. The presence of the cuts 124 interrupts strain in the material that could be present in a simple linear slit 112. The presence of the cuts 124 may allow for easier insertion or passing of the at least one fastening element 116 by allowing an inherent flexibility to create an open gate in the visor 102.

The use of the slits 112 enables the at least one fastening element 116 to pass through the visor 102 with minimal damage to structural integrity. Advantageously, the use of slits 112 rather than holes with a greater three-dimensional presence increases ease of manufacturing and scalability of the visor 102. Furthermore, earloops 116 of disposable face masks 120 commonly in use could not pass through holes in the visor 102. The use of slits 112 accommodates multiple different types of fastening elements 116, including both individual cords or strings 116 as well as earloops 116.

The use of the at least one fastening element 116 of the face mask 120 to secure the visor 102 further increases both the integrity of the assembled visor assembly 100 and the ease with which the visor 102 may be removed from the assembled visor assembly 100 so that the face mask 120 may be used alone. Because the at least one fastening element 116 of the face mask 120 are used to secure the visor 102, the visor 102 cannot be removed and cannot fall off while the visor assembly 100 is in use on the wearer's face 134 and head 144. For example, the earloops 116 that secure the face mask 120 also secure the visor 102. Unlike conventional surgical goggles or glasses, the visor 102 remains securely positioned on the wearer's face 134. However, as soon as the visor assembly 100 is removed from the wearer's face 134, the wearer 140 may separate the visor 102 from the face mask 120 by simply pulling the at least one fastening element 116 (for example, earloops 116) back through the slits 112.

A further advantage of this method of attachment is the adjustability of the visor 102 while the visor assembly 100 is in place. The wearer 140 of the visor assembly 100 may adjust the visor 102 up or down the wearer's own face 134 and may further increase or decrease an angle between the visor 102 and the wearer's own face 134. This adjustment occurs through pivoting and/or sliding the visor 102 about the at least one fastening element 116 while the at least one fastening element 116 are passed through the slits 112. Where the at least one fastening element 116 are properly secured as they would be if the wearer 140 were wearing the mask 120 alone without the visor 102 (for example, in the case of earloops 116 properly affixed about the wearer's own ears 154), this adjustment may advantageously not affect the integrity of the visor assembly 100 and the overall position of the face mask 120 on the wearer's own face 134. The slits 112 may be sufficiently long so as to accommodate wearers 140 with larger heads 144 and/or larger faces 134 and further may offer adjustability of positioning of the visor 102 and consequently the visor assembly 100 for all wearers 140.

The slits 112 may be substantially straight and parallel to the first surface 104 (i.e., vertical, as shown in FIG. 2). The slits 112 instead may be substantially straight and perpendicular to the first surface 104 (i.e., horizontal compared to the slits 112 of FIG. 2). The slits 112 may be substantially straight and be at any other angle relative to the first surface 104. These configurations may promote ease of manufacturing. These configurations may further promote ease of assembly of the visor assembly 100 by the wearer 140. Any of these configurations may allow for easier insertion or passing of the at least one fastening element 116 by allowing an inherent flexibility to create an open gate in the visor 102.

The slits 112 may be substantially curved and may be convex relative to an interior side edge of each member 110. The slits 112 may instead be substantially curved and may be concave relative to an interior side edge of each member 110. These configurations may promote ease of manufacturing. These configurations may further promote ease of assembly of the visor assembly 100 by the wearer 140. This configuration may allow for a rigid configuration that positions the visor assembly 100 further away from the wearer's eyes 136 without being positioned too far down the wearer's nose 138.

The slits 112 may include cuts 124, which may be at the top and/or the bottom of the slits 112 as shown in FIG. 1, or elsewhere along slits 112. Cuts 124 may advantageously help retain the at least one fastening element 116 in place within the slits 112.

The visor 102 may be sufficiently flexible so as to mold to the wearer's own face 134. The visor 102 may deform no more than necessary for the at least one fastening element 116 to remain substantially straight. Alternately, the visor 102 may be relatively rigid even when in use such that the visor 102 deforms no more than necessary in order for the at least one fastening element 116 to remain substantially straight. When not in use, the visor 102 may assume its original shape.

The visor 102 may be entirely made of transparent material, such as an optically clear plastic or other plastic. Alternately, only the first surface 104 of the visor 102 may be made of such transparent material. Advantageously, this configuration may reduce glare for the wearer 140 (e.g., while performing surgery) and/or may reduce ambient light (e.g., when outdoors). The first surface 104 includes a viewing area 132 through which the wearer may see past the visor 102.

Distance 128 represents the distance from the top of the slit 112 to the bottom of the second surface 106 of the visor 102. As shown in FIG. 2, the slit 112 may extend beyond the member 110 upwards into the second surface 106. The slit 112 may even extend beyond the member 110 and second surface 106 upwards into the first surface 104. Such different lengths of the distance 128 and/or of the slits 112 may accommodate wearers 140 with different preferences and/or physical requirements of the head 144 and/or face 134. A longer slit 112 may accommodate different sizes of face 134 of wearers 140. Such accommodation may advantageously reducing stretching and breaking of the at least one fastening element 116.

Distance 130 represents the distance that the members 110 extend past the second surface 106 of the visor 102. Such different lengths of the distance 138 and/or of the members 110 may accommodate wearers 140 with different preferences and/or physical requirements of the head 144 and/or face 134. Such extension of the members 110 may advantageously reduce stretching and breaking of the at least one fastening element 116 when passing through the slits 112.

Such reduced stretching and breaking of the at least one fastening element 116 upon passage through the slits 112 may advantageously cause the mask 120 and accordingly the visor assembly 100 to more securely and easily remain in place on the face 134 and about the head 144 of the wearer 140, thereby better protecting the wearer 140.

Advantageously, the visor 102 may be capable of cleaning and reuse after separation from the mask 120 to effect disassembly of the visor assembly 100. By contrast, conventional means of attaching the visor 102 to the mask 120, such as welding, rather than the use of fastening elements 116 and slits 112 as herein described, are not susceptible to such cleaning and reuse as the visor 102 may accordingly not be easily separable from the mask 120.

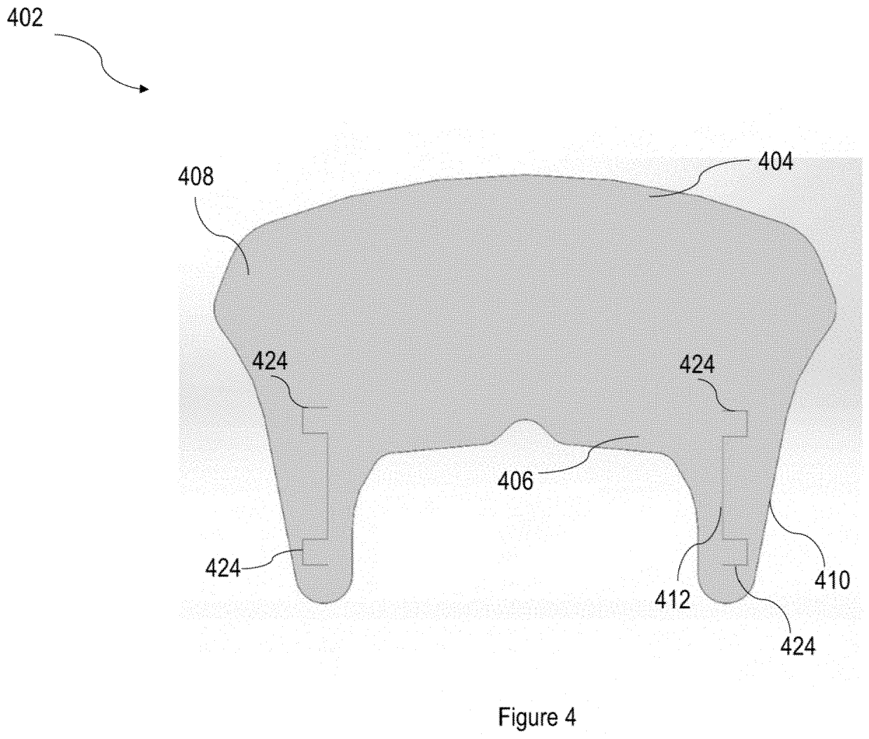

Referring now to FIG. 4, shown therein is an alternate embodiment 402 of the visor 102 of FIGS. 1-3. In FIG. 4, like references denote like features.

The visor 402 includes first surface 404, second surface 406, and third surfaces 408 as in the visor 102. The visor 402 further includes members 410, which include slits 412. The wider members 410 may advantageously provide greater structural integrity to the visor 402 and visor assembly 400.

The first surface 404 of the visor 402 may advantageously extend higher up the face 434 of the wearer 440 (visible as a curved upper edge of the first surface 404 in FIG. 4) than does first surface 104 of visor 102 in FIG. 1. The higher first surface 404 may advantageously protect more of the wearer's face 434. The higher first surface 404 may advantageously accommodate larger heads 444 and/or faces 434 of wearers 440, particularly taller heads 444 and/or faces 434.

The outer edges of third surfaces 408 define a compound angle relative to the first surface 404, in contradiction to the outer edges of first surface 108, which define only a single angle relative to the first surface 104. That the third surfaces 408 are accordingly less pronounced and less sharp may advantageously allow the visor 402 to be safer for wearers 440 to handle and wear and for storage purposes. The compound angle formed by third surface 408 arises because the third surfaces 408 are formed in part by two protrusions relative to the first surface 404, whereas the third surfaces 108 are formed through a single protrusion relative to the first surface 104.

The slits 412 include cuts 424. The cuts 424 are cut in a rectangular shape. This shape may advantageously better hold the at least one fastening element 416 in place. The cuts 424 may also be cut such that the slits 412 form a D′ shape in the members 410.

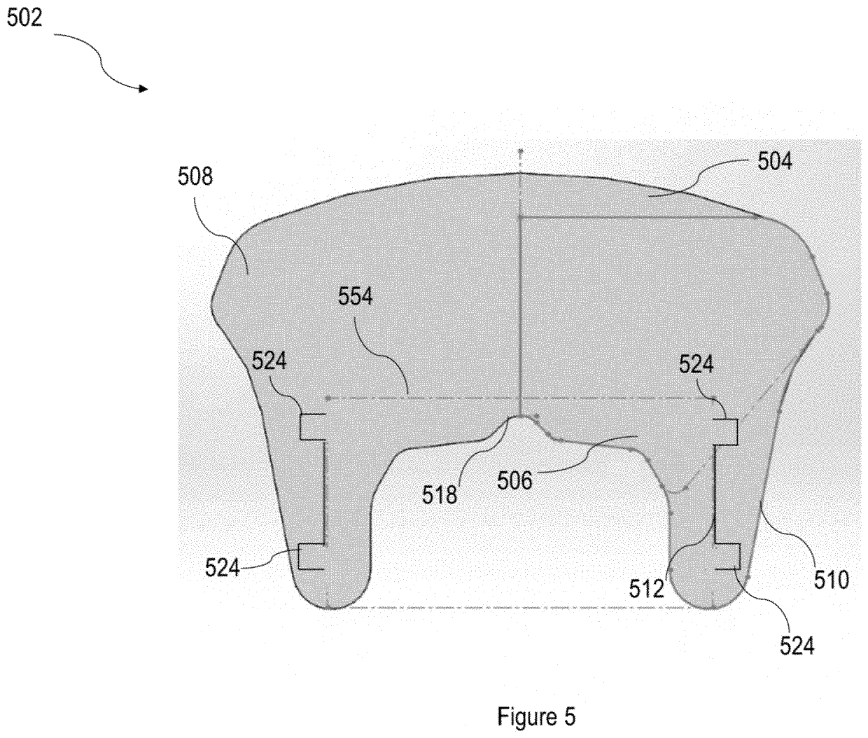

Referring now to FIG. 5, shown therein is an alternate embodiment 502 of the visor 102 of FIGS. 1-4. In FIG. 5, like references denote like features.

The visor 502 includes first surface 504, second surface 506, and third surfaces 508 as in the visor 102. The visor 502 further includes members 510, which include slits 512.

The visor 502 further includes narrowed distance 554 between the slits 512 across the wearer's face 534. This narrowed distance 554 may cause the visor 502 to advantageously sit on the pleats of the mask 520 and hold the mask 520 in place on the face 534 of the wearer 540. With distance 554 between the slits 512 and consequently the members 510, the members 510 may advantageously hold the sides of the mask 520 in place in order to create a better seal between the mask 520 and the wearer 540, thereby advantageously improving the functionality of the mask 520 in the visor assembly 500.

In the visor 502, the region 518 may sit more comfortably or efficaciously on the nose 538 of the wearer.

The visor 502 includes slits 512 that are lower in the visor 502 than are the slits 112 in the visor 102. Such lower slits 512 may advantageously more easily engage the at least one fastening element 516 of the mask 520 to secure the visor 502 in place.

The slits 512 include cuts 524. The cuts 524 are cut in a rectangular shape. This shape may advantageously better hold the at least one fastening element 516 in place. The cuts 524 may also be cut such that the slits 512 form a D′ shape in the members 510.

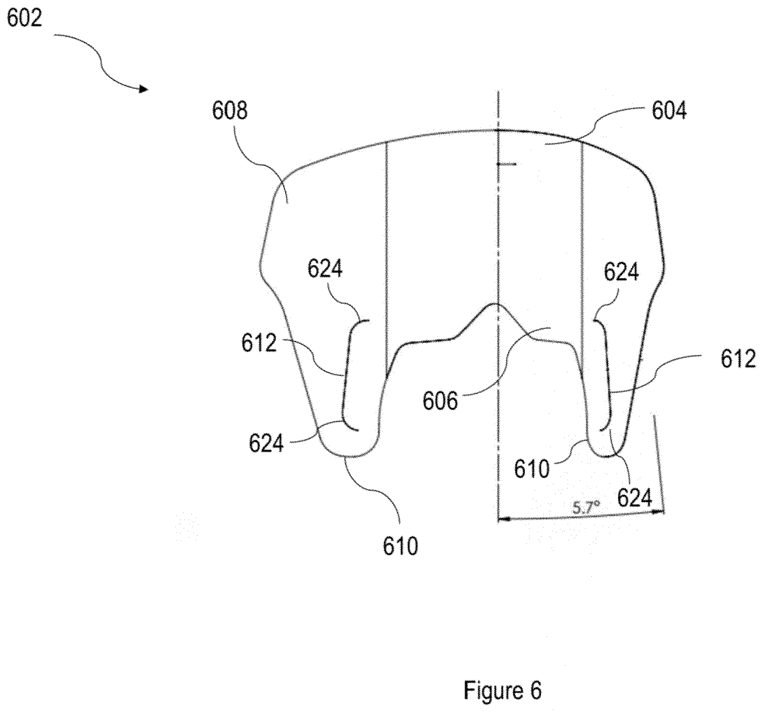

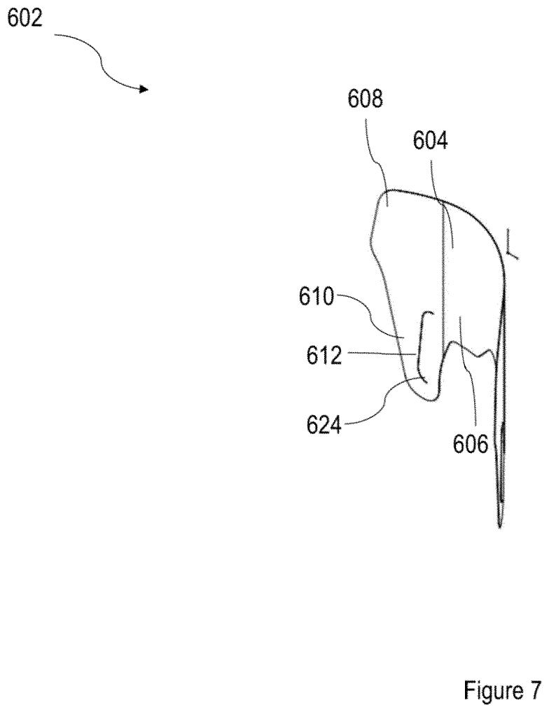

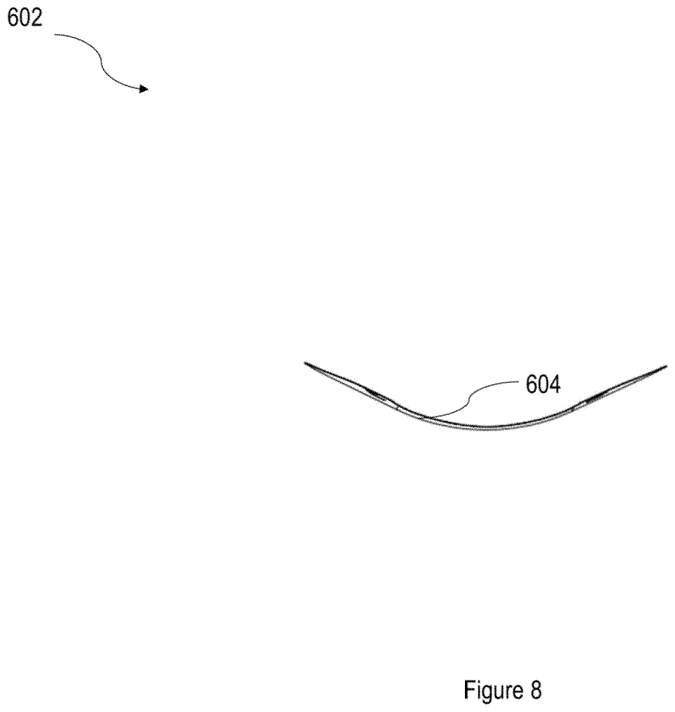

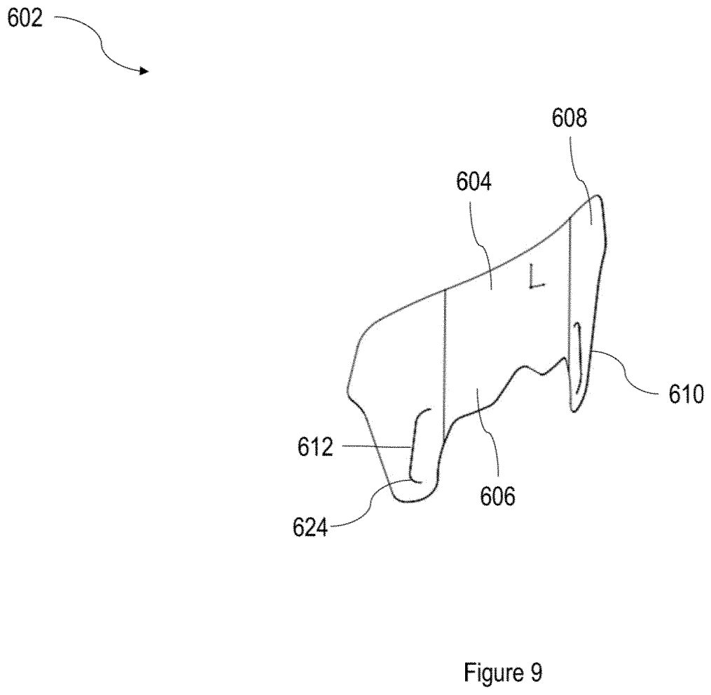

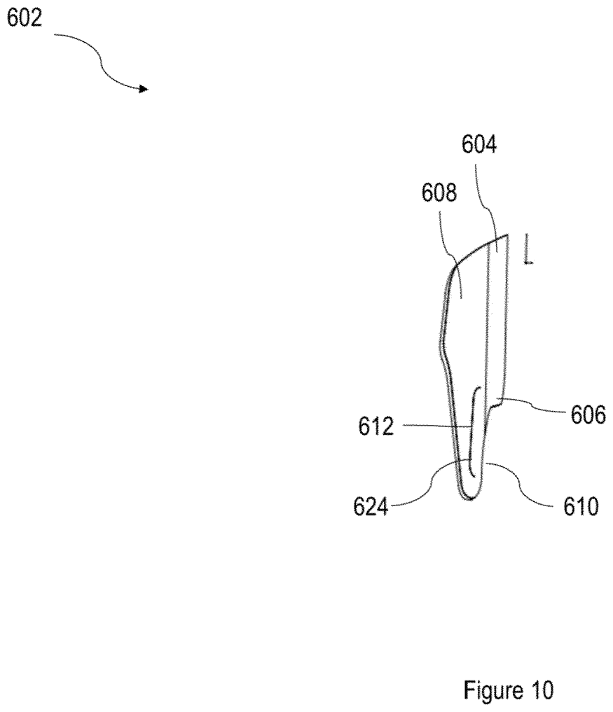

Referring now to FIGS. 6 to 10, shown therein is an alternate embodiment 602 of the visor 102 of FIGS. 1-5. In FIGS. 6 to 10, like references denote like features.

The visor 602 includes first surface 604, second surface 606, and third surfaces 608 as in the visor 102. The visor 602 further includes members 610, which further include slits 612.

In the visor 602, the members 610 and slits 612 are canted outwards, away from the first, second, and third surfaces 604, 606, and 608. Accordingly, the internal edges of the members 610 may be closer to each other than in previous embodiments. Such a configuration of the members 610 and slits 612 may advantageously increase the stability of the visor 602 and associated visor assembly 600 and may further allow the visor 602 to be worn further from the wearer's own face 634. Such a configuration may further advantageously permit the wearer 640 to comfortably wear glasses or other eyewear over the eyes 636 and about the face 634.

The slits 612 include cuts 624. The cuts 624 may be cut in a rectangular shape. This shape may advantageously better hold the at least one fastening element 616 in place.

In FIG. 6, the cuts 624 are in the shape of curves contiguous with the slits 612. This shape of the cuts 624 may advantageously allow for easy repositioning of fastening elements 616 by the wearer 640. The cuts 624 may be cut such that the slits 612 form a D′ shape in the members 610.

The internal edges of the members 610 of the visor 602 are closer to each other in visor 602 and so may keep the sides of the mask 620 pressed against the face 634 of the wearer 640. In order to achieve this, the members 610 are canted on a dihedral angle of less than 10 degrees (for example, 5.7 degrees in FIG. 6) with respect to a vertical axis through the centre of the visor 602. For ease of manufacture, the cuts 624 may not be in a D′ or rectangular shape.

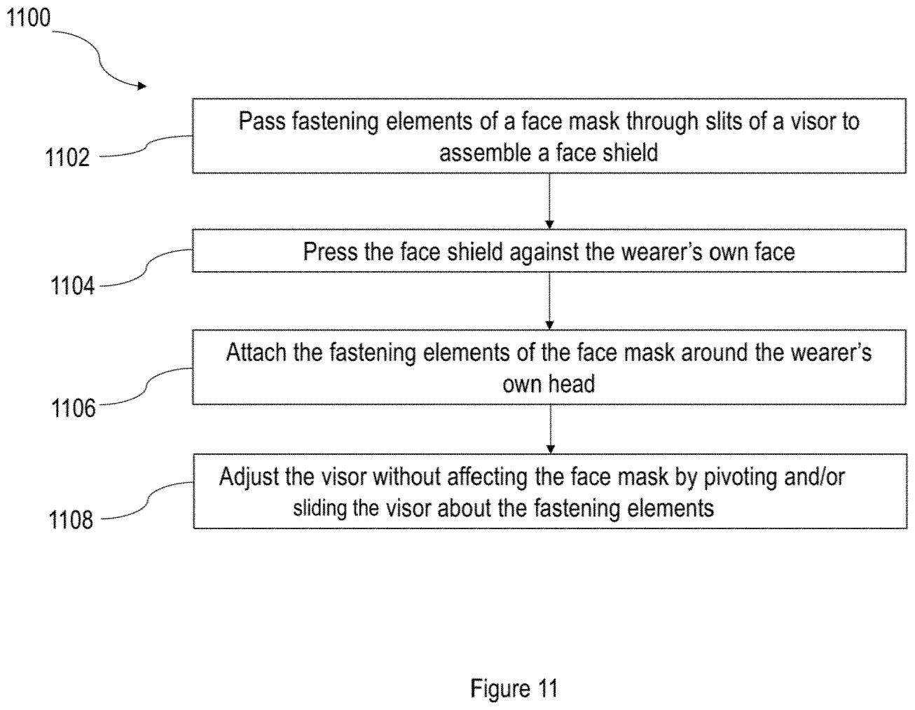

Referring now to FIG. 11, shown therein is a method 1100 of shielding a wearer's face from biological hazards.

At 1102, the wearer passes the at least one fastening element 116 of the face mask 120 through the slits 112 of the visor 102 (or visor 402, 502, 602) to assemble the visor assembly 100. For example, the wearer may pass the earloops 116 shown in FIG. 1 through the slits 112 shown in FIGS. 1-3. Advantageously, the top and bottom of the earloops 116 may rest in each of the cuts 124, which may be upper and lower cuts 124, respectively, on each side of the visor assembly 100. The cuts 124 may ensure that top and bottom portions of the earloops 116 do not tangle or otherwise migrate to the middle of the slit 112, which might cause discomfort for the wearer 140 and might result in the face mask 120, the visor 102, and/or the entire visor assembly 100 not remaining on the wearer's face 134 and/or about the wearer's head 144.

The two protruding members 110 of the visor 102 protrude substantially in parallel to the first surface 104 of the visor 102 towards the disposable face mask 120. Each member 110 includes at least one slit 112 through which one of the at least one fastening element 116 of the disposable face mask 120 may pass at least once.

At 1104, the wearer 140 presses the assembled visor assembly 100 against their own face 134.

At 1106, the wearer 140 attaches the at least one fastening element 116 of the face mask 120 around the wearer's own head 144.

At 1108, the wearer may adjust the visor 102 without affecting the fit of the face mask 120 by pivoting and/or sliding the visor 102 about the at least one fastening element 116.

Advantageously, the visor 102 may be capable of cleaning and reuse after separation from the mask 120 to effect disassembly of the visor assembly 100. By contrast, conventional means of attaching the visor 102 to the mask 120, such as welding, rather than the use of fastening elements 116 and slits 112 as herein described, are not susceptible to such cleaning and reuse as the visor 102 may accordingly not be easily separable from the mask 120.

Advantageously, the visor 102 may be made of a recyclable material.

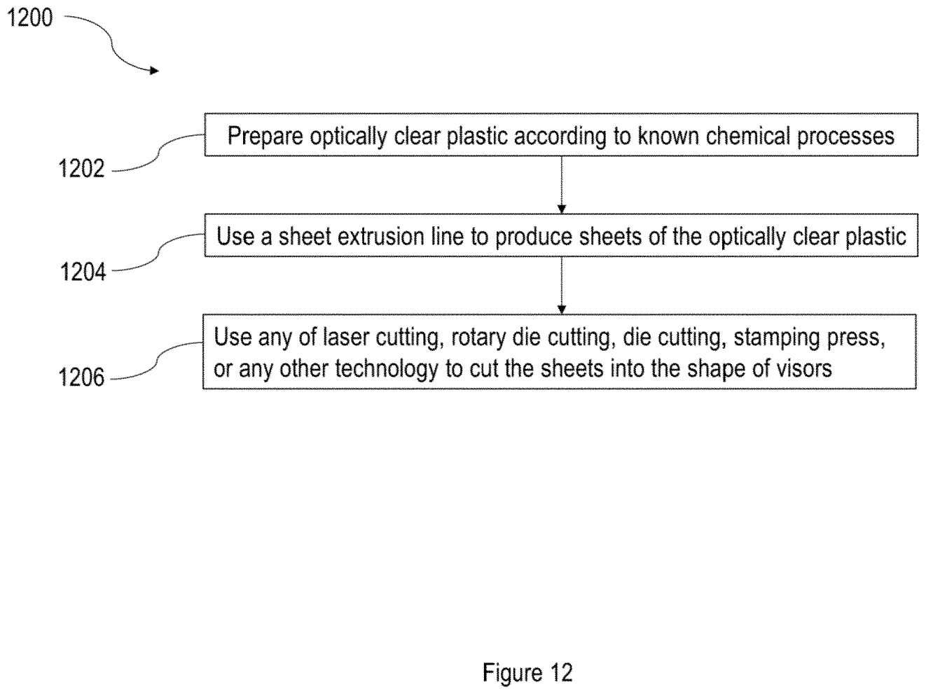

Referring now to FIG. 12, shown therein is a method 1200 of manufacturing a visor 102 (or visor 402, 502, 602) configured to connect to a disposable face mask 120.

At 1202, preparing optically clear plastic according to known chemical processes.

At 1204, using a sheet extrusion line to produce sheets of the optically clear plastic.

At 1206, using any of laser cutting, rotary die cutting, die cutting, stamping press, or any other technology to cut the sheets into the shape of the visors 102.

In the case of a die cutting process, in order to increase efficiency of the process, multiple sheets of the optically clear plastic may be stamped at a time.

The plastic sheet may include a protective lamination affixed to one or both exterior sides. The lamination may be peeled off before wearing the visor 102. The lamination may help prevent the visor 102 from being scratched, for example in transit. The lamination may help prevent contamination of the visor 102 before use.

Advantageously, the visor 102 may be capable of cleaning and reuse after separation from the mask 120 to effect disassembly of the visor assembly 100. By contrast, conventional means of attaching the visor 102 to the mask 120, such as welding, rather than the use of fastening elements 116 and slits 112 as herein described, are not susceptible to such cleaning and reuse as the visor 102 may accordingly not be easily separable from the mask 120.

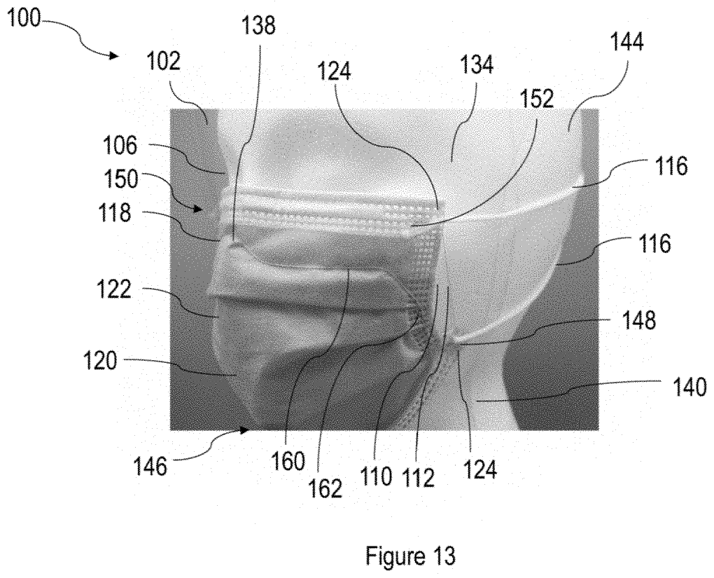

Referring now to FIG. 13, shown therein is an enlarged view of part of the visor assembly 100 of FIG. 1.

In FIG. 13, the integration of the at least one fastening element 116 (in FIG. 13, earloops 116) of the mask 120 with the visor 102 (or visor 402, 502, 602) may be more clearly seen. To effect this integration, the wearer 140 (in FIG. 13, mannequin 140) passes the at least one fastening element 116 through the slits 112 of the members 110 of the visor 102. The slits 112 include cuts 124, which may be at the top and/or bottom of the slits 112 as shown, or elsewhere along slits 112. In the case of the visor 102 shown in FIG. 13, the cuts 124 are in the form of curves in which the at least one fastening element 116 may be lodged. The cuts 124 may also take the form of rectangular cuts at the top and bottom of the slits 112. The cuts 124 may also be cut such that the slits 112 form a D′ shape in the members 110.

The at least one fastening element 116 are attached to the mask 120 at each of the corners 146, 148, 150, and 152 In FIG. 1, corners 150 and 146 of the mask 120 were visible, while corners 152 and 148 were not. In FIG. 13, because the wearer 140 is depicted facing to the left rather than to the right of the image, corners 152 and 148 are visible, while corners 150 and 146 are located on the other side of the mask 120 about the face 134 of the mannequin 140.

In FIG. 13, the cutout region 118 of the mask body 122 is properly supported by the the nose 138 of the wearer 140.

In FIG. 13, the lower portion 160 of the second surface 106 adjacent to the cutout region 118 and the inner portion 162 of the member 110 may provide a functional improvement to the mask 120. This function improvement may arise through increasing the efficacy of the seal between the mask 120 and the wearer's face 134. Because the weight and pressure of the visor 102 as exerted through the lower portion 160 of the second surface 106 and the inner portion 162 of the member 110 are focused on the mask body 122, sides of the mask 120 may advantageously be pressed against the wearer's face 134. This functional improvement may further increase the health and safety of the wearer 140, thereby improving the mask 120.

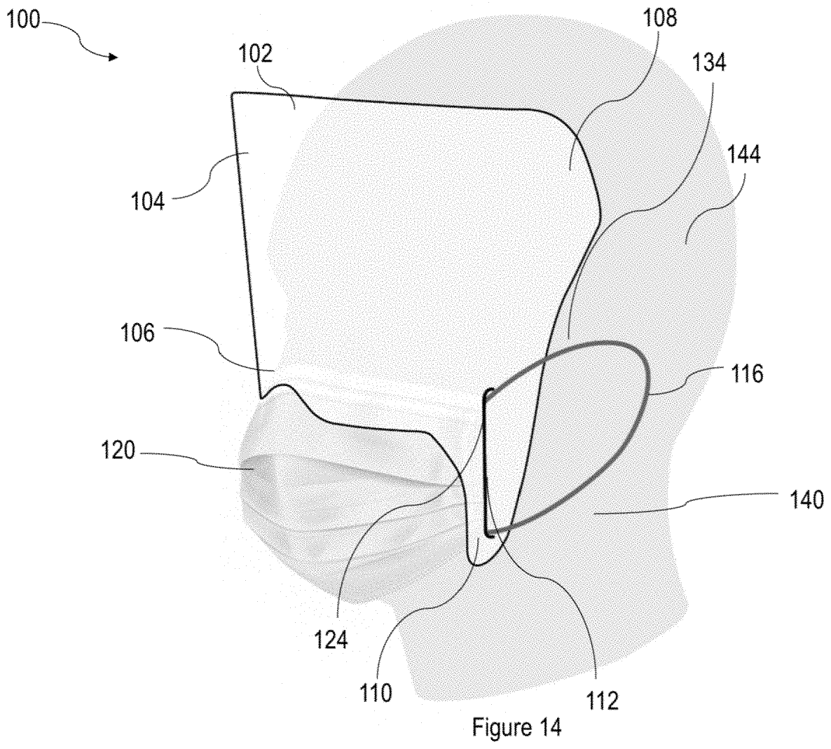

Referring now to FIG. 14, shown therein is another view of the visor assembly 100 of FIGS. 1 and 13. Like references denote like features.

It will be appreciated by those skilled in the art that, where a series of actions are described in the context of a method or process, those actions may be taken in any order that produces the desired result. Examples of a method being illustrated or performed are not to be taken as restricting the scope of any methods or processes described or claimed to the exact series or order of actions herein described or claimed.

While the above description provides examples of one or more apparatus, methods, or systems, it will be appreciated that other apparatus, methods, or systems may be within the scope of the claims as interpreted by one of skill in the art.

1. A visor assembly to be worn as personal protective equipment about a wearer's face, the visor assembly comprising:
a visor configured to connect to a disposable face mask, the visor comprising: a first surface that is substantially flat when the visor is not connected to the disposable face mask, wherein the first surface is substantially curved when the visor is connected to the disposable face mask and worn; a second surface that is substantially flat when the visor is not connected to the disposable face mask, wherein the second surface is substantially curved when the visor is connected to the disposable face mask and worn; two protruding members that protrude substantially in parallel to the first surface towards the disposable face mask, each member comprising at least one slit through which at least one fastening element of the disposable face mask may pass at least once; and
a face mask comprising the at least one fastening element, wherein the at least one fastening element is passed through the slits of the members of the visor.

2. The visor assembly of claim 1, wherein when in use the visor is sufficiently flexible to mold to the wearer's face, wherein when in use the visor deforms no more than necessary for the at least one fastening element to remain substantially straight, wherein when not in use the visor assumes its original shape, and wherein the visor is made of transparent material.

3. The visor assembly of claim 1, wherein only the first surface of the visor is made of transparent material.

4. The visor assembly of claim 1, wherein the second surface comprises a cutout region to accommodate a wearer's nose.

5. The visor assembly of claim 4, wherein the cutout region is sufficiently flexible to fold around the wearer's nose.

6. The visor assembly of claim 1, wherein the visor further comprises third surfaces adjacent to the first surface that wrap around sides of the wearer's face when the visor is in use.

7. The visor assembly of claim 1, wherein the visor is made of a recyclable material.

8. The visor assembly of claim 1, wherein the visor may be separated from the visor assembly and cleaned for reuse.

9. The visor assembly of claim 6, wherein the third surfaces comprise a compound angle, wherein the first surface comprises a dome portion as an upper edge, wherein the members are canted outwards, and wherein the members rest atop the mask.

10. The visor assembly of claim 1, wherein the slits further comprise at least one cut to hold the at least one fastening element in place.

11. A kit comprising the visor and face mask of claim 1.

12. A visor configured to connect to a disposable face mask to be worn about a wearer's face, the visor comprising:
a first surface that is substantially flat when the visor is not connected to the disposable face mask, wherein the first surface is substantially curved when the visor is connected to the disposable face mask and worn;
a second surface that is substantially flat when the visor is not connected to the disposable face mask, wherein the second surface is substantially curved when the visor is connected to the disposable face mask and worn;
two protruding members that protrude substantially in parallel to the first surface towards the disposable face mask, each member comprising at least one slit through which at least one fastening element of the disposable face mask may pass at least once.

13. The visor of claim 12, wherein when in use the visor is sufficiently flexible to mold to the wearer's face, wherein when in use the visor deforms no more than necessary for the at least one fastening element to remain substantially straight, wherein when not in use the visor assumes its original shape, and wherein the visor is made of transparent material.

14. The visor of claim 12, wherein only the first surface of the visor is made of transparent material.

15. The visor of claim 12, wherein the second surface comprises a cutout region to accommodate a wearer's nose.

16. The visor of claim 15, wherein the cutout region is sufficiently flexible to fold around the wearer's nose.

17. The visor of claim 12, wherein the visor further comprises third surfaces adjacent to the first surface that wrap around sides of the wearer's face when the visor is in use.

18. The visor of claim 12, wherein the visor is made of a recyclable material.

19. The visor of claim 12, wherein the visor may be separated from the disposable face mask and cleaned for reuse.

20. The visor of claim 17, wherein the third surfaces comprise a compound angle, wherein the first surface comprises a dome portion as an upper edge, wherein the members are canted outwards, and wherein the members rest atop the mask.
